(19) United States
(12) Patent Application Publication
(10) Pub. No.: US 2025/0104242 A1
(43) Pub. Date: Mar. 27, 2025

(54) MEDICAL SUPPORT DEVICE, ENDOSCOPE APPARATUS, MEDICAL SUPPORT SYSTEM, MEDICAL SUPPORT METHOD, AND PROGRAM

(71) Applicant: FUJIFILM CORPORATION, Tokyo (JP)

(72) Inventor: Daisuke NAKAJIMA, Kanagawa (JP)

(21) Appl. No.: 18/830,507

(22) Filed: Sep. 10, 2024

(30) Foreign Application Priority Data
Sep. 26, 2023 (JP) 2023-163551

(51) Int. Cl. G06T 7/00; A61B 1/00; G06V 10/77; G16H 40/67

(52) U.S. Cl. CPC G06T 7/0016; A61B 1/000094; A61B 1/0005; G06V 10/77; G16H 40/67; G06T 2207/10068; G06T 2207/30096; G06T 2207/30204; G06V 2201/03

(57) ABSTRACT

A medical support device includes a communication device and a processor. The communication device transmits a first medical image to a processing apparatus and receives a processing result obtained by executing processing on the first medical image by the processing apparatus. The processor performs a first control of outputting time information related to a time taken from transmission of the first medical image to the processing apparatus by the communication device to reception of the processing result, and/or a second control of making an output aspect of the processing result different depending on the time.

### CROSS-REFERENCE TO RELATED APPLICATIONS

This application claims priority under 35 USC 119 from Japanese Patent Application No. 2023-163551, filed on Sep. 26, 2023, the entire disclosure of which is incorporated by reference herein.

### BACKGROUND

### 1. Technical Field

The present disclosure relates to a medical support device, an endoscope apparatus, a medical support system, a medical support method, and a program.

### 2. Related Art

JP2011-036488A discloses an endoscope system including an electronic endoscope that images an observation target to generate an image signal, a processor to which the electronic endoscope is attachably and detachably connected, and an image processing apparatus that is attachably and detachably connected to the processor. In the endoscope system disclosed in JP2011-036488A, the image processing apparatus comprises a first image processing unit that performs first image processing on the image signal to generate a first video signal, and a processing time output unit that outputs information related to a processing time in the first image processing to the processor. In the electronic endoscope system disclosed in JPJP2011-036488A, the processor comprises an image signal output unit that outputs the image signal to the image processing apparatus, a second image processing unit that performs second image processing on the image signal to generate a second video signal, and a first timing controller that controls a timing of outputting the second video signal based on the information related to the processing time.

JP2022-142990A discloses an examination support device that is connected to an endoscope system comprising a camera unit to be inserted into a body of a subject and is used. The examination support device disclosed in JP2022-142990A comprises an acquisition unit, a detection unit, a diagnosis unit, and a display controller. In the examination support device disclosed in JP2022-142990A, the acquisition unit sequentially acquires image signals provided to an external device by the endoscope system. The detection unit detects whether or not a frame image sequentially generated from the image signal is a still image or a moving image. In a case where the detection unit detects that the frame image is the still image, the diagnosis unit inputs a diagnosis image obtained by cutting out an image region of at least a part of the frame image to a trained model to perform a diagnosis inside the body. The display controller displays a diagnosis result output by the diagnosis unit on a display unit.

In the examination support device disclosed in JP2022-142990A, the display controller does not display the diagnosis result until a preset retention time elapses from a time when the detection unit detects that the frame image is the still image, and even in a case where the retention time has elapsed, the display controller does not display the diagnosis result in a case where the detection unit detects that the frame image generated from the image signal acquired by the acquisition unit at a point in time when the retention time has elapsed is the moving image.

### SUMMARY

One embodiment of the present disclosure provides a medical support device, an endoscope apparatus, a medical support system, a medical support method, and a program capable of allowing a user to recognize a degree of a time lag caused by transmission and reception between the medical support device and a processing apparatus.

A first aspect according to the present disclosure is a medical support device comprising: a communication device; and a processor, in which the communication device transmits a first medical image to a processing apparatus, and receives a processing result obtained by executing processing on the first medical image by the processing apparatus, and the processor performs a first control of outputting time information related to a time taken from transmission of the first medical image to the processing apparatus by the communication device to reception of the processing result, and/or a second control of making an output aspect of the processing result different depending on the time.

A second aspect according to the present disclosure is the medical support device according to the first aspect, in which the outputting of the time information by the processor includes displaying the time information on a first screen by the processor.

A third aspect according to the present disclosure is the medical support device according to the second aspect, in which the first control includes a control of displaying the first medical image and/or a second medical image obtained temporally later than the first medical image on a second screen in a state in which the first medical image and/or the second medical image is comparable with the time information.

A fourth aspect according to the present disclosure is the medical support device according to any one of the first to third aspects, in which the time information includes first specifying information for specifying the time itself.

A fifth aspect according to the present disclosure is the medical support device according to any one of the first to fourth aspects, in which the time information includes second specifying information for specifying a difference between the time and a first reference time.

A sixth aspect according to the present disclosure is the medical support device according to the fifth aspect, in which the second specifying information includes length information for specifying a length of the time with respect to the first reference time.

A seventh aspect according to the present disclosure is the medical support device according to any one of the first to sixth aspects, in which the time information is visualized information.

An eighth aspect according to the present disclosure is the medical support device according to the seventh aspect, in which the time information is information visualized by a text and/or a mark.

A ninth aspect according to the present disclosure is the medical support device according to the seventh or eighth aspect, in which the processor changes a visualization format, which is a format in which the time information is visualized, according to the time.

A tenth aspect according to the present disclosure is the medical support device according to the ninth aspect, in which the processor makes visibility of the time information in a case where the time is equal to or longer than a second reference time higher than visibility of the time information in a case where the time is shorter than the second reference time.

An eleventh aspect according to the present disclosure is the medical support device according to any one of the first to tenth aspects, in which the output aspect is a display aspect in which the processor displays the processing result on a third screen.

A twelfth aspect according to the present disclosure is the medical support device according to the eleventh aspect, in which the display aspect includes a first display aspect and a second display aspect, the first display aspect is used in a case where the time is equal to or longer than a third reference time and is used to specify that the time is equal to or longer than the third reference time, and the second display aspect is used in a case where the time is shorter than the third reference time and is used to specify that the time is shorter than the third reference time.

A thirteenth aspect according to the present disclosure is the medical support device according to the twelfth aspect, in which the second display aspect has higher visibility than the first display aspect.

A fourteenth aspect according to the present disclosure is the medical support device according to the thirteenth aspect, in which the processing result is not displayed in the first display aspect, and the processing result is displayed in the second display aspect.

A fifteenth aspect according to the present disclosure is the medical support device according to any one of the eleventh to fourteenth aspects, in which the second control includes a control of displaying the first medical image and/or a third medical image obtained temporally later than the first medical image on a fourth screen in a state in which the first medical image and/or the third medical image is comparable with the processing result.

A sixteenth aspect according to the present disclosure is the medical support device according to any one of the eleventh to fourteenth aspects, in which the second control includes a control of displaying the first medical image on a fourth screen in a state in which the first medical image is comparable with the processing result in a case where the time is shorter than a fourth reference time.

A seventeenth aspect according to the present disclosure is the medical support device according to the sixteenth aspect, in which the fourth reference time is a longest time within which a degree of affinity between the first medical image and the processing result falls within an allowable range.

An eighteenth aspect according to the present disclosure is the medical support device according to any one of the first to seventeenth aspects, in which the processing is an AI process for the first medical image.

A nineteenth aspect according to the present disclosure is the medical support device according to any one of the first to eighteenth aspects, in which the processing includes a recognition process of recognizing a feature region shown in the first medical image.

A twentieth aspect according to the present disclosure is the medical support device according to the nineteenth aspect, in which the feature region is a lesion.

A twenty-first aspect according to the present disclosure is the medical support device according to any one of the first to twentieth aspects, in which the first medical image is an endoscopic image generated by imaging an inside of a body by a camera mounted on an endoscope.

A twenty-second aspect according to the present is an endoscope apparatus comprising: the medical support device according to any one of the first to twenty-first aspects; and a camera that is inserted into a body and generates the first medical image by imaging an inside of the body.

A twenty-third aspect according to the present disclosure is a medical support system comprising: a medical support device; and a processing apparatus, in which the medical support device transmits a first medical image to the processing apparatus, the processing apparatus executes processing on the first medical image and transmits a processing result obtained by executing the processing to the medical support device, and the medical support device receives the processing result transmitted from the processing apparatus, and performs a first control of outputting time information related to a time taken from transmission of the first medical image to the processing apparatus to reception of the processing result by the medical support device, and/or a second control of making an output aspect of the processing result different depending on the time.

A twenty-fourth aspect according to the present disclosure is a medical support method comprising: transmitting a first medical image to a processing apparatus; receiving a processing result obtained by executing processing on the first medical image by the processing apparatus; and performing a first control of outputting time information related to a time taken from transmission of the first medical image to the processing apparatus to reception of the processing result, and/or a second control of making an output aspect of the processing result different depending on the time.

A twenty-fifth aspect according to the present disclosure is a program for causing a computer to execute a medical support process comprising: transmitting a first medical image to a processing apparatus; receiving a processing result obtained by executing processing on the first medical image by the processing apparatus; and performing a first control of outputting time information related to a time taken from transmission of the first medical image to the processing apparatus to reception of the processing result, and/or a second control of making an output aspect of the processing result different depending on the time.

### BRIEF DESCRIPTION OF THE DRAWINGS

Exemplary embodiments of the technology of the disclosure will be described in detail based on the following figures, wherein:

FIG. 1 is a conceptual diagram showing an example of an aspect in which a medical support system is used by a doctor;

FIG. 2 is a conceptual diagram showing an example of an overall configuration of the medical support system;

FIG. 3 is a block diagram showing an example of a hardware configuration of an electric system of the medical support system;

FIG. 4 is a block diagram showing an example of functions of main units of a processor included in a medical support device and an example of information stored in a storage;

FIG. 5 is a conceptual diagram showing an example of processing contents of the medical support device and a processing apparatus;

FIG. 6 is a conceptual diagram showing an example of processing contents of a controller and an example of a content displayed on a screen of a display device;

FIG. 7 is a flowchart showing an example of a flow of a medical support process;

FIG. 8 is a conceptual diagram showing a modification example of a content displayed in a second display region in a case where a transmission/reception time is equal to or longer than a reference time;

FIG. 9 is a conceptual diagram showing a modification example of the content displayed on the screen of the display device; and

FIG. 10 is a conceptual diagram showing a display example of a case where a frame obtained temporally later than a frame to be displayed in a first display region in a case where the transmission/reception time is shorter than the reference time is used as a frame to be displayed in the first display region in a case where the transmission and reception time is equal to or longer than the reference time.

### DETAILED DESCRIPTION

Hereinafter, examples of embodiments of a medical support device, an endoscope apparatus, a medical support system, a medical support method, and a program according to the present disclosure will be described with reference to the accompanying drawings.

First, terms that are used in the following description will be described.

CPU refers to an abbreviation of a “Central Processing Unit”. GPU is an abbreviation for a “graphics processing unit”. GPGPU is an abbreviation for “general-purpose computing on graphics processing units”. APU is an abbreviation for an “accelerated processing unit”. TPU is an abbreviation for a “tensor processing unit”. RAM is an abbreviation for a “random access memory”. NVM is an abbreviation for a “non-volatile memory”. EEPROM is an abbreviation for an “electrically erasable programmable read-only memory”. ASIC is an abbreviation for an “application specific integrated circuit”. PLD is an abbreviation for a “programmable logic device”. FPGA is an abbreviation for a “field-programmable gate array”. SoC is an abbreviation for a “system-on-a-chip”. SSD is an abbreviation for a “solid state drive”. USB is an abbreviation for a “universal serial bus”. HDD is an abbreviation for a “hard disk drive”. EL is an abbreviation for “electro-luminescence”. CMOS is an abbreviation for a “complementary metal oxide semiconductor”. CCD is an abbreviation for a “charge coupled device”. AI is an abbreviation for “artificial intelligence”. BLI is an abbreviation for “blue light imaging”. LCI is an abbreviation for “linked color imaging”. I/F is an abbreviation for an “Interface”. SSL stands for a “sessile serrated lesion”. LAN is an abbreviation for a “local area network”. WAN is an abbreviation for a “wide area network”. 5G is an abbreviation for a “5th generation mobile communication system”. RTC stands for “Real Time Clock”. IC is an abbreviation for “Integrated Circuit”. MRI is an abbreviation of “magnetic resonance imaging”. CT is an abbreviation of “computed tomography”.

In the following description, a processor with a reference (hereinafter, simply referred to as a “processor”) may be one physical or virtual computing device or a combination of a plurality of physical or virtual computing devices. In addition, the processor may be one type of a computing device or a combination of a plurality of types of computing devices. Examples of the computing device include a CPU, a GPU, a GPGPU, an APU, or a TPU.

In the following description, a memory with a reference is a memory such as a RAM in which information is temporarily stored, and is used as a work memory by the processor.

In the following description, a storage with a reference is one or a plurality of non-volatile storage devices that store various programs, various parameters, and the like. Examples of the non-volatile storage device include a flash memory, a magnetic disk, or a magnetic tape. In addition, other examples of the storage include a cloud storage.

In the following embodiment, an external I/F with a reference transmits and receives various types of information between a plurality of devices connected to each other. Examples of the external I/F include a USB interface. A communication I/F including a communication processor, an antenna, and the like may be applied to the external I/F. The communication I/F performs communication between a plurality of computers. Examples of a communication standard applied to the communication I/F include a wireless communication standard including 5G, Wi-Fi (registered trademark), or Bluetooth (registered trademark).

In the following embodiments, “A and/or B” is synonymous with “at least one of A or B”. That is, “A and/or B” means that it may be only A, only B, or a combination of A and B. In addition, in the present specification, in a case where three or more matters are associated and represented by “and/or”, the same concept as “A and/or B” is applied.

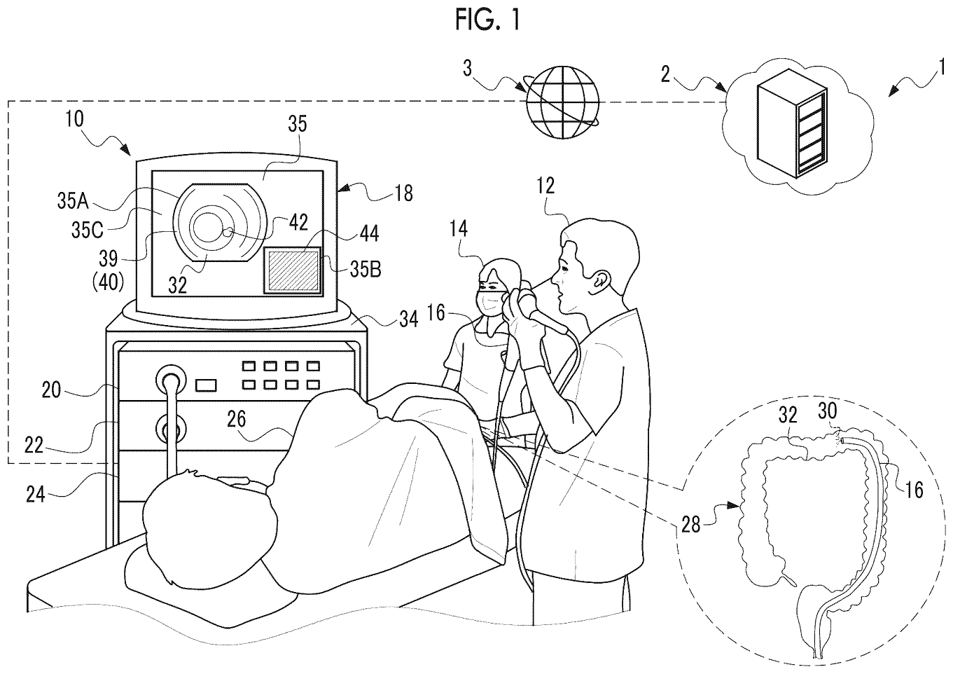

FIG. 1 is a conceptual diagram showing an example of an aspect in which a medical support system 1 is used. As shown in FIG. 1, the medical support system 1 comprises a processing apparatus 2 and an endoscope apparatus 10, and the processing apparatus 2 and the endoscope apparatus 10 are communicably connected to each other via a network 3. Examples of the network 3 include the Internet. However, the Internet is merely an example, and examples of the other network 3 include a WAN and/or a LAN. In the present embodiment, the medical support system 1 is an example of a “medical support system” according to the present disclosure, and the processing apparatus 2 is an example of a “processing apparatus” according to the present disclosure.

The endoscope apparatus 10 is used by a doctor 12 in endoscopy and the like. The endoscopy is assisted by a staff such as a nurse 14.

Information obtained by the endoscope apparatus 10 is transmitted to the processing apparatus 2 via the network 3. Examples of the processing apparatus 2 include a cloud server. However, the cloud server is merely an example, and the processing apparatus 2 may be an on-premises server or a personal computer. The processing apparatus 2 receives the information transmitted from the endoscope apparatus 10, executes processing using the received information, and transmits a processing result obtained by executing the processing to the endoscope apparatus 10.

The endoscope apparatus 10 comprises an endoscope 16, a display device 18, a light source device 20, a control device 22, and a medical support device 24. In the present embodiment, the endoscope 16 is an example of an “endoscope” according to the present disclosure, the endoscope apparatus 10 is an example of an “endoscope apparatus” according to the present disclosure, and the medical support device 24 is an example of a “medical support device” according to the present disclosure.

The endoscope apparatus 10 is a modality for performing medical care on a large intestine 28 included in a body of a subject 26 (for example, a patient) by using the endoscope 16. In the present embodiment, the large intestine 28 is a target to be observed by the doctor 12.

The endoscope 16 is used by the doctor 12 and is inserted into the body of the subject 26. In the present embodiment, the endoscope 16 is inserted into the large intestine 28 which is a luminal organ of the subject 26.

The endoscope apparatus 10 causes the endoscope 16 inserted into the large intestine 28 of the subject 26 to image the inside of the large intestine 28 of the subject 26 and performs various medical treatments on the large intestine 28 as necessary.

The endoscope apparatus 10 acquires and outputs an image showing an aspect in the large intestine 28 by imaging the inside of the large intestine 28 of the subject 26. In the present embodiment, the endoscope apparatus 10 is an endoscope apparatus having an optical imaging function of capturing reflected light obtained by being reflected by an intestinal wall 32 of the large intestine 28 by irradiating the inside of the large intestine 28 with light 30.

Although the endoscopy of the large intestine 28 is illustrated here, this is merely an example, and the present disclosure is established even in a case of an endoscopy of a luminal organ such as an esophagus, a stomach, a duodenum, or a trachea.

The light source device 20, the control device 22, and the medical support device 24 are installed on a wagon 34. A plurality of tables are provided in the wagon 34 along a vertical direction, and the medical support device 24, the control device 22, and the light source device 20 are installed from a lower table to an upper table. In addition, the display device 18 is installed on the uppermost table in the wagon 34.

The control device 22 controls the entire endoscope apparatus 10. The medical support device 24 performs various types of image processing on an image obtained by imaging the intestinal wall 32 with the endoscope 16 under the control of the control device 22. In addition, the medical support device 24 is communicably connected to the processing apparatus 2 via the network 3, and requests the processing apparatus 2 to provide a service, thereby receiving the requested service from the processing apparatus 2.

The display device 18 displays various types of information including the image. Examples of the display device 18 include a liquid crystal display or an EL display. In addition, a tablet terminal with a display may be used instead of the display device 18 or together with the display device 18.

A screen 35 is displayed on the display device 18. The screen 35 includes a plurality of display regions. The plurality of display regions are arranged side by side in the screen 35. In the example shown in FIG. 1, as an example of the plurality of display regions, a first display region 35A, a second display region 35B, and a third display region 35C are shown. A size of the first display region 35A is larger than a size of the second display region 35B. The first display region 35A is used as a main display region, and the second display region 35B is used as a sub-display region. A size relationship between the first display region 35A and the second display region 35B is not limited to this, and need only be a size relationship that fits on the screen 35. The third display region 35C is a display region other than the first display region 35A and the second display region 35B in the screen 35, and is used as a sub-display region. In the present embodiment, the screen 35 is an example of a “first screen”, a “second screen”, a “third screen”, and a “fourth screen” according to the present disclosure.

An endoscopic moving image 39 is displayed in the first display region 35A. The endoscopic moving image 39 is a moving image acquired by imaging the intestinal wall 32 with the endoscope 16 inside the large intestine 28 of the subject 26. In the example shown in FIG. 1, as an example of the endoscopic moving image 39, a moving image in which the intestinal wall 32 is shown is shown.

The intestinal wall 32 shown in the endoscopic moving image 39 includes a lesion 42 (for example, one lesion 42 in the example shown in FIG. 1,) as a region of interest (that is, an observation target region) that is watched by the doctor 12, and the doctor 12 can visually recognize an aspect of the intestinal wall 32 including the lesion 42 through the endoscopic moving image 39. In the present embodiment, the lesion 42 is an example of a “feature region” and a “lesion” according to the present disclosure.

The lesion 42 has various types, and examples of the type of the lesion 42 include a neoplastic polyp and a non-neoplastic polyp. Examples of the type of the neoplastic polyp include an adenomatous polyp (for example, SSL). Examples of the type of the non-neoplastic polyp include a hamartomatous polyp, a hyperplastic polyp, and an inflammatory polyp. The types illustrated here are types assumed in advance as the types of the lesion 42 in a case where the endoscopy is performed on the large intestine 28, and the types of the lesion 42 may be different depending on the organ on which the endoscopy is performed.

In the present embodiment, for convenience of description, a form example is described in which one lesion 42 is shown in the endoscopic moving image 39, but the present disclosure is not limited to this. The present disclosure is established even in a case where a plurality of the lesions 42 are shown in the endoscopic moving image 39.

In the present embodiment, the lesion 42 is illustrated, but this is merely an example. The region of interest (that is, the observation target region) that is watched by the doctor 12 may be a feature region having some unique feature, such as an organ (for example, a duodenal papilla), a mark, an artificial treatment tool (for example, an artificial clip), a treated region (for example, a region in which a trace of removal of a polyp or the like remains), or the like.

The image displayed in the first display region 35A is one frame 40 included in a moving image configured to include a plurality of frames 40 along a time series. That is, a plurality of frames 40 along the time series are displayed in the first display region 35A at a predetermined frame rate (for example, several tens of frames/second).

Examples of the moving image displayed in the first display region 35A include a moving image of a live view method. The live view method is only an example, and a moving image which is temporarily stored in a memory or the like and then is displayed, such as a moving image of a post view method, may be employed. In addition, each frame included in a recording moving image stored in a memory or the like may be reproduced and displayed on the screen 35 (for example, the first display region 35A) as the endoscopic moving image 39.

In the screen 35, the second display region 35B is adjacent to the first display region 35A and is displayed in the lower right in the screen 35 in front view. A display position of the second display region 35B may be anywhere in the screen 35 of the display device 18. However, the display position is preferably displayed at a position comparable with the endoscopic moving image 39.

In the second display region 35B, assistance information 44 for assisting the doctor 12 in medical determination or the like in the endoscopy is displayed. The assistance information 44 is information referred to by the doctor 12. Examples of the assistance information 44 include various types of information about the subject 26 in which the endoscope 16 is inserted into the body, and/or various types of information obtained by performing a medical support process described below.

Visible information obtained by performing processing using AI on the frame 40 is displayed in the third display region 35C. Details thereof will be described later.

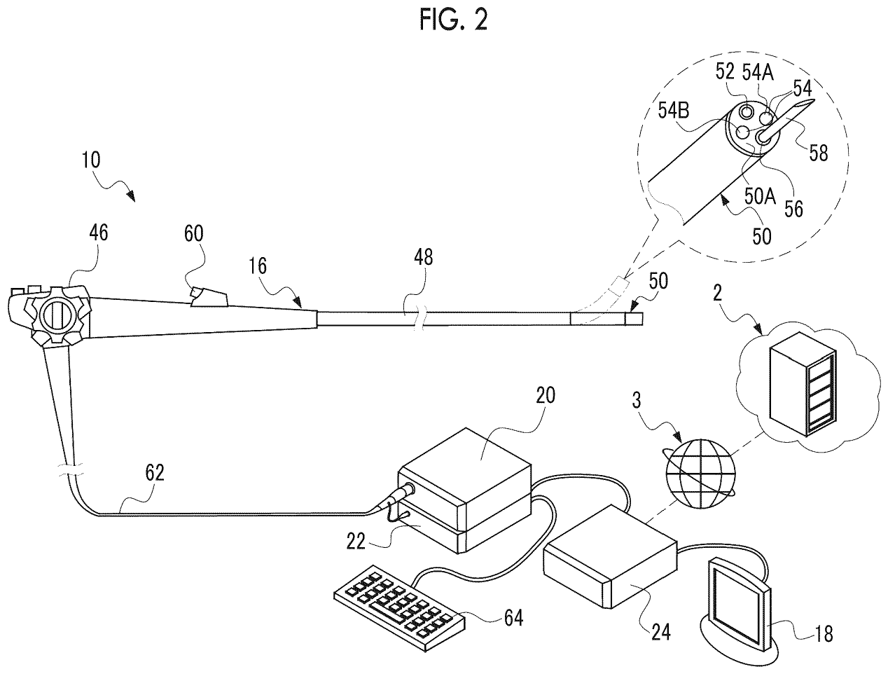

FIG. 2 is a conceptual diagram showing an example of an overall configuration of the endoscope apparatus 10. As shown in FIG. 2, the endoscope 16 comprises an operating part 46 and an insertion part 48. The insertion part 48 is partially bent by operating the operating part 46. The insertion part 48 is inserted into the large intestine 28 while being bent according to a shape of the large intestine 28 (see FIG. 1) in response to the operation of the operating part 46 by the doctor 12 (see FIG. 1).

A camera 52, an illumination device 54, and a treatment tool opening 56 are provided in a distal end part 50 of the insertion part 48. The camera 52 and the illumination device 54 are provided on a distal end surface 50A of the distal end part 50. Here, although a form example is described in which the camera 52 and the illumination device 54 are provided on the distal end surface 50A of the distal end part 50, this is merely an example. The camera 52 and the illumination device 54 may be provided on a side surface of the distal end part 50, so that the endoscope 16 may be configured as a side-viewing endoscope.

The camera 52 is mounted on the endoscope 16 and is inserted into a body cavity of the subject 26 to image the observation target region to generate the frame 40 as an endoscopic image. In the present embodiment, the camera 52 generates the endoscopic moving image 39 including the plurality of frames 40 along the time series by imaging the inside of the body (for example, the inside of the large intestine 28) of the subject 26. Examples of the camera 52 include a CMOS camera. However, this is only an example, and the camera may be other types of camera such as a CCD camera. In the present embodiment, the camera 52 is an example of a “camera” according to the present disclosure, and the frame 40 is an example of a “first medical image”, a “second medical image”, a “third medical image”, and an “endoscopic image” according to the present disclosure.

The illumination device 54 has illumination windows 54A and 54B. The illumination device 54 emits the light 30 (see FIG. 1) through illumination windows 54A and 54B. Examples of the type of the light 30 emitted from the illumination device 54 include visible light (for example, white light) and invisible light (for example, near-infrared light). In addition, the illumination device 54 emits special light through the illumination windows 54A and 54B. Examples of the special light include light for BLI and/or light for LCI. The camera 52 images the inside of the large intestine 28 by an optical method in a state in which the light 30 is emitted inside the large intestine 28 by the illumination device 54.

The treatment tool opening 56 is an opening through which a treatment tool 58 protrudes from the distal end part 50. In addition, the treatment tool opening 56 is also used as a suction port for sucking blood, internal filth, and the like and a delivery port for sending out a fluid.

A treatment tool insertion port 60 is formed in the operating part 46, and the treatment tool 58 is inserted into the insertion part 48 through the treatment tool insertion port 60. The treatment tool 58 passes through the insertion part 48 and protrudes from the treatment tool opening 56 to the outside. In the example shown in FIG. 2, an aspect is shown in which a puncture needle protrudes from the treatment tool opening 56 as the treatment tool 58. Here, the puncture needle is illustrated as the treatment tool 58, but this is merely an example. The treatment tool 58 may be a grasping forceps, a papillotomy knife, a snare, a catheter, a guide wire, a cannula, and/or a puncture needle with a guide sheath, or the like.

The endoscope 16 is connected to the light source device 20 and the control device 22 via a universal cord 62. The medical support device 24 and a reception device 64 are connected to the control device 22. In addition to the processing apparatus 2, the display device 18 is also connected to the medical support device 24. That is, the control device 22 is connected to the processing apparatus 2 and the display device 18 via the medical support device 24.

Here, since the medical support device 24 is illustrated as an externally connected device for expanding a function performed by the control device 22, a form example is described in which the control device 22 and the display device 18 are indirectly connected to each other via the medical support device 24, but this is merely an example. For example, the display device 18 may be directly connected to the control device 22. In this case, for example, functions of the medical support device 24 may be mounted on the control device 22.

The reception device 64 receives an instruction from the doctor 12 and outputs the received instruction as an electric signal to the control device 22. Examples of the reception device 64 include a keyboard, a mouse, a touch panel, a foot switch, a microphone, and/or a remote control device.

The control device 22 controls the light source device 20, transmits and receives various signals to and from the camera 52, or transmits and receives various signals to and from the medical support device 24.

The light source device 20 emits light under the control of the control device 22 and supplies the light to the illumination device 54. A light guide is provided in the illumination device 54, and the light supplied from the light source device 20 is emitted from the illumination windows 54A and 54B through the light guide. The light emitted from the illumination windows 54A and 54B is the light 30 (see FIG. 1). The control device 22 causes the camera 52 to perform imaging, acquires the endoscopic moving image 39 (see FIG. 1) from the camera 52, and outputs the endoscopic moving image 39 to a predetermined output destination (for example, the medical support device 24).

The medical support device 24 supports medical care (here, as an example, an endoscopy) by performing various types of image processing on the endoscopic moving image 39 input from the control device 22. The medical support device 24 outputs the endoscopic moving image 39 that has been subjected to various types of image processing to a predetermined output destination (for example, the display device 18).

Here, a form example is described in which the endoscopic moving image 39 output from the control device 22 is output to the display device 18 via the medical support device 24, but this is merely an example. For example, the control device 22 and the display device 18 may be connected to each other, and the endoscopic moving image 39 that has been subjected to the image processing by the medical support device 24 may be displayed on the display device 18 via the control device 22.

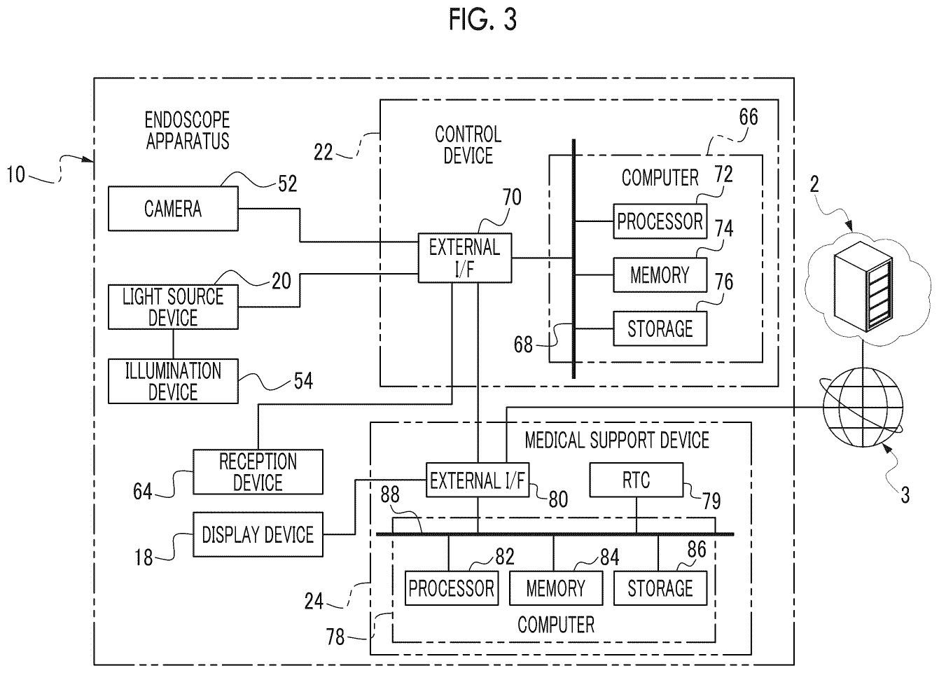

FIG. 3 is a block diagram showing an example of a hardware configuration of an electric system of the endoscope apparatus 10. As shown in FIG. 3, the control device 22 comprises a computer 66, a bus 68, and an external I/F 70. The computer 66 comprises a processor 72, a memory 74, and a storage 76. The processor 72, the memory 74, the storage 76, and the external I/F 70 are connected to the bus 68. The processor 72 controls the entire control device 22. The memory 74 and the storage 76 are used by the processor 72.

The external I/F 70 transmits and receives various types of information between one or more devices (hereinafter, also referred to as “first external devices”) outside the control device 22 and the processor 72.

As one of the first external devices, the camera 52 is connected to the external I/F 70, and the external I/F 70 transmits and receives various types of information between the camera 52 and the processor 72. The processor 72 controls the camera 52 via the external I/F 70. In addition, the processor 72 acquires the endoscopic moving image 39 (see FIG. 1) obtained by imaging the inside of the large intestine 28 (see FIG. 1) by the camera 52 via the external I/F 70.

As one of the first external devices, the light source device 20 is connected to the external I/F 70, and the external I/F 70 transmits and receives various types of information between the light source device 20 and the processor 72. The light source device 20 supplies light to the illumination device 54 under the control of the processor 72. The illumination device 54 performs irradiation with the light supplied from the light source device 20.

As one of the first external devices, the reception device 64 is connected to the external I/F 70. The processor 72 acquires the instruction received by the reception device 64 via the external I/F 70 and performs a process corresponding to the acquired instruction.

The medical support device 24 comprises a computer 78, an RTC 79, and an external I/F 80. The computer 78 comprises a processor 82, a memory 84, and a storage 86. The processor 82, the memory 84, the storage 86, the RTC 79, and the external I/F 80 are connected to a bus 88. In the present embodiment, the computer 78 is an example of a “computer” according to the present disclosure, and the processor 82 is an example of a “processor” according to the present disclosure.

Since a hardware configuration (that is, the processor 82, the memory 84, and the storage 86) of the computer 78 is basically the same as the hardware configuration of the computer 66, the hardware configuration of the computer 78 will not be described here.

The RTC 79 is an IC that generates and outputs data such as a current time point. The processor 82 acquires the data such as the current time point from the RTC 79. Here, although a form example in which the processor 82 acquires the data of the current time point from the RTC 79 is described, this is merely an example, and the processor 82 may acquire the data of the current time point from a server or the like on the network 3.

The external I/F 80 transmits and receives various types of information between one or more devices (hereinafter, also referred to as “second external devices”) outside the medical support device 24 and the processor 82.

As one of the second external devices, the control device 22 is connected to the external I/F 80. In the example shown in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscopic moving image 39 (see FIG. 1) from the processor 72 of the control device 22 via the external I/Fs 70 and 80 and performs various types of image processing on the acquired endoscopic moving image 39.

As one of the second external devices, the display device 18 is connected to the external I/F 80. The processor 82 controls the display device 18 via the external I/F 80 so that various types of information (for example, the endoscopic moving image 39 subjected to various types of image processing) are displayed on the display device 18.

The processing apparatus 2 is connected to the external I/F 80 via the network 3 as one of the second external devices. The processor 82 exchanges various kinds of information with the processing apparatus 2 via the external I/F 80. For example, the external I/F 80 transmits the endoscopic moving image 39 acquired from the camera 52 to the processing apparatus 2 by the processor 82. The processing apparatus 2 receives the endoscopic moving image 39 transmitted from the external I/F 80, executes processing using at least one frame 40 included in the received endoscopic moving image 39, and transmits a processing result to the medical support device 24. The external I/F 80 receives the processing result transmitted from the processing apparatus 2. The processor 82 acquires the processing result received by the external I/F 80. In the present embodiment, the external I/F 80 is an example of a “communication device” according to the present disclosure.

Meanwhile, in recent years, a technology has been developed in which a trained model optimized by performing machine learning on a model (for example, a neural network) performs an object recognition process on each frame 40 included in the endoscopic moving image 39 to recognize the lesion 42, and a recognition result of the object recognition process or information based on the recognition result of the object recognition process is displayed on the screen 35.

However, the object recognition process performed on each frame 40 included in the endoscopic moving image 39 has a very large processing load. Therefore, currently, a technology is being studied in which the endoscope apparatus 10 causes the processing apparatus 2 communicably connected to the endoscope apparatus 10 via the network 3 to execute the object recognition process and to transmit the processing result of the object recognition process to the endoscope apparatus 10 to thereby reduce the processing load on the endoscope apparatus 10.

In a case where this technology is used, a time lag due to transmission and reception between the endoscope apparatus 10 and the processing apparatus 2 (hereinafter, also simply referred to as a “time lag”) occurs. In a case where the time lag occurs, there is a concern that a content of the frame 40 displayed in the first display region 35A of the endoscope apparatus 10 does not match the content of the processing result by the processing apparatus 2 (for example, the recognition result of the object recognition process or the information based on the recognition result of the object recognition process). In addition, in a case where the time lag is extremely short, the content of the frame 40 displayed in the first display region 35A of the endoscope apparatus 10 substantially matches the content of the processing result by the processing apparatus 2. Therefore, the time lag does not have a large influence on the diagnosis by the doctor 12.

In a case where the object recognition process is executed by the processing apparatus 2 communicably connected to the endoscope apparatus 10 via the network 3, it is very important for the doctor 12 to recognize how long the time lag is in order for the doctor 12 to make a correct diagnosis in endoscopy. In addition, this is not limited to the endoscopy, and is also applicable to other types of medical examinations (for example, a radiography examination, an MRI examination, or a CT examination).

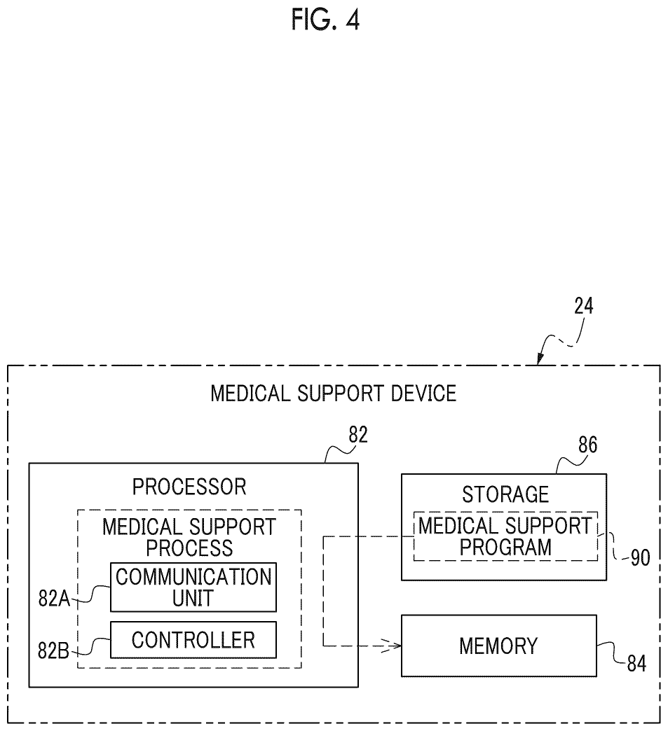

Therefore, in view of such circumstances, in the present embodiment, as shown in FIG. 4 as an example, the medical support process is performed by the processor 82 of the medical support device 24. FIG. 4 is a block diagram showing an example of functions of main units of the processor 82 included in the medical support device 24 and an example of information stored in the storage 86.

A medical support program 90 is stored in the storage 86. The medical support program 90 is an example of a “program” according to the present disclosure. The processor 82 reads out the medical support program 90 from the storage 86 and executes the read-out medical support program 90 on the memory 84 to perform the medical support process. The medical support process is realized by the processor 82 operating as a communication unit 82A and a controller 82B according to the medical support program 90 executed on the memory 84.

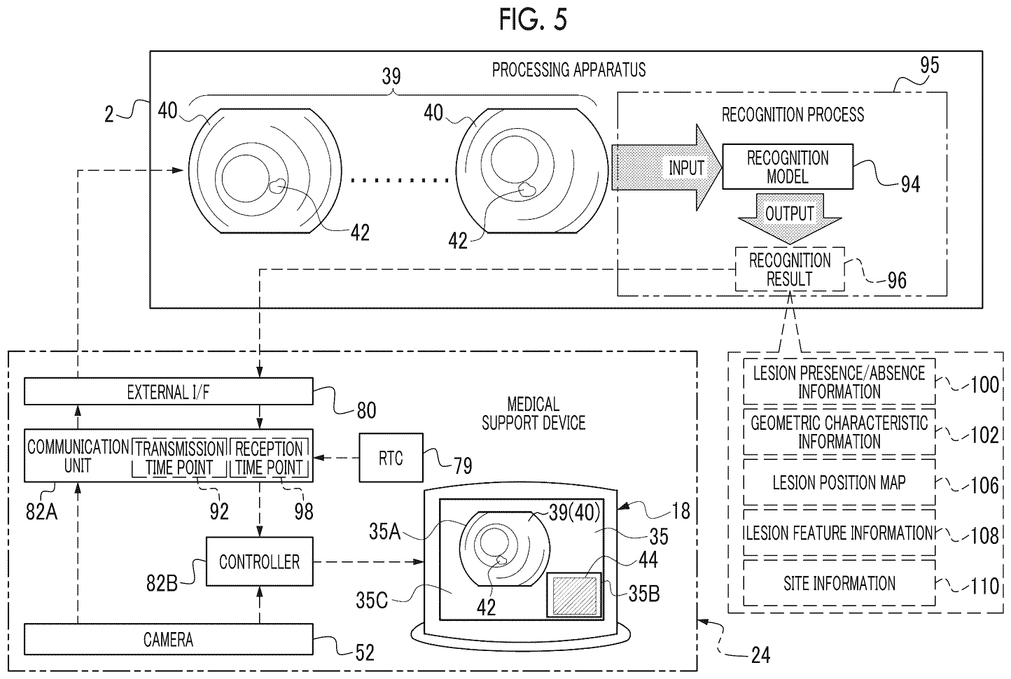

FIG. 5 is a conceptual diagram showing an example of the processing contents performed by the medical support device 24 and the processing apparatus 2. As shown in FIG. 5, the communication unit 82A and the controller 82B acquire each of the plurality of frames 40 along the time series included in the endoscopic moving image 39, which is generated by being captured by the camera 52 at an imaging frame rate (for example, several tens of frames/second), from the camera 52 in units of one frame along the time series. Timings at which the communication unit 82A and the controller 82B acquire the frame 40 are synchronized.

The controller 82B outputs the endoscopic moving image 39 to the display device 18. For example, the controller 82B displays the endoscopic moving image 39 in the first display region 35A as a live view image. That is, each time the frame 40 is acquired from the camera 52, the controller 82B displays the acquired frame 40 in the first display region 35A in order at a display frame rate (for example, several tens of frames/second). In addition, the controller 82B displays the assistance information 44 in the second display region 35B. In addition, the controller 82B updates the display content (for example, the assistance information 44) of the second display region 35B according to the display content of the first display region 35A.

The communication unit 82A performs communication (that is, transmission and reception of various kinds of information) with the processing apparatus 2 via the external I/F 80 (see FIG. 3). The communication unit 82A transmits the endoscopic moving image 39 acquired from the camera 52 to the processing apparatus 2 via the external I/F 80 (see FIG. 3). Here, the communication unit 82A acquires a transmission time point 92 that is a time point at which the endoscopic moving image 39 is transmitted to the processing apparatus 2. The transmission time point 92 is, for example, a time point acquired from the RTC 79 by the communication unit 82A at a timing at which the endoscopic moving image 39 is transmitted from the external I/F 80 to the processing apparatus 2.

The processing apparatus 2 receives the endoscopic moving image 39 transmitted from the communication unit 82A. The processing apparatus 2 has a recognition model 94 and executes a recognition process 95 using the recognition model 94 on the endoscopic moving image 39. The processing apparatus 2 may be a device that only executes the recognition process 95 or may be a device that executes a plurality of processes including the recognition process 95. For example, the plurality of processes may include a process of performing processing on at least one frame 40 included in the endoscopic moving image 39. In the present embodiment, the recognition process 95 is an example of the object recognition process described above and is an example of an “AI process” and a “recognition process” according to the present disclosure.

The processing apparatus 2 recognizes the lesion 42 in the endoscopic moving image 39 based on the endoscopic moving image 39. That is, the processing apparatus 2 recognizes the lesion 42 shown in the frame 40 by sequentially performing the recognition process 95 on each of the plurality of frames 40 along the time series included in the endoscopic moving image 39 acquired from the camera 52.

For example, the processing apparatus 2 recognizes geometric characteristics (for example, a position, a size, and a shape) of the lesion 42, a kind of the lesion 42, a type of the lesion 42 (for example, a pedunculated type, a subpedunculated type, a sessile type, a surface raised type, a surface flat type, a surface depressed type, and the like), and the like. Further, the processing apparatus 2 recognizes a site in the endoscopic moving image 39 (hereinafter, simply referred to as a “site”) based on the endoscopic moving image 39 acquired from the camera 52. Here, the site refers to a site of the large intestine 28. Examples of the site of the large intestine 28 include an anal canal, a lower rectum, an upper rectum, a sigmoid colon, a descending colon, a transverse colon, an ascending colon, and an ileocecal portion.

The recognition process 95 is performed on the acquired frame 40 each time the frame 40 is acquired by the processing apparatus 2. The recognition process 95 is a process of recognizing the lesion 42 and the site by a method using AI. Here, as the recognition process 95, a process using the recognition model 94 is performed.

The recognition model 94 is a trained model for object recognition in a bounding box method using AI. The recognition model 94 is optimized by performing machine learning on a neural network using training data. The training data is a data set including a plurality of data (that is, a plurality of frames of data) in which example data and correct answer data are associated with each other.

The example data is an image assuming the frame 40. First examples of the image assuming the frame 40 include an image obtained by actually imaging the inside of the large intestine with the camera. Second examples of the image assuming the frame 40 include an image virtually created (for example, an image generated by generation AI). The correct answer data is correct answer data (that is, an annotation) for the example data. Here, as an example of the correct answer data, an annotation for specifying geometric characteristics of a lesion shown in an image used as the example data, a kind of the lesion, a type of the lesion, and a site is used.

The processing apparatus 2 acquires the frame 40 from the camera 52 and inputs the acquired frame 40 to the recognition model 94. As a result, the recognition model 94 recognizes the lesion 42 and the site shown in the input frame 40 each time the frame 40 is input, and generates and outputs a recognition result 96. In the present embodiment, the recognition result 96 is an example of a “processing result” according to the present disclosure.

The processing apparatus 2 transmits the recognition result 96 itself to the medical support device 24 or transmits information generated based on the recognition result 96 to the medical support device 24. In the medical support device 24, the external I/F 80 receives the recognition result 96 transmitted from the processing apparatus 2. The communication unit 82A acquires the recognition result 96 received by the external I/F 80.

Here, the communication unit 82A acquires a reception time point 98 that is a time point at which the endoscopic moving image 39 is received by the external I/F 80. The reception time point 98 is, for example, a time point acquired by the communication unit 82A from the RTC 79 at a timing at which the recognition result 96 transmitted from the processing apparatus 2 is received by the external I/F 80.

The controller 82B acquires the recognition result 96, the transmission time point 92, and the reception time point 98 from the communication unit 82A. The controller 82B displays the recognition result 96 itself on the screen 35 (for example, the first display region 35A, the second display region 35B, and/or the third display region 35C) of the display device 18 or displays the information generated based on the recognition result 96 on the screen 35 (for example, the first display region 35A, the second display region 35B, and/or the third display region 35C) based on the recognition result 96, the transmission time point 92, and the reception time point 98.

The recognition result 96 includes lesion presence/absence information 100. The lesion presence/absence information 100 is information indicating whether or not the lesion 42 is shown in the frame 40 input to the recognition model 94. In addition, the recognition result 96 includes geometrical characteristic information 102, a lesion position map 106, lesion feature information 108, and the like.

The geometric characteristic information 102 is information (for example, coordinates) for specifying a shape, a size, and a position of the lesion 42 in the frame 40. The geometric characteristic information 102 or the information based on the geometric characteristic information 102 may be displayed in the first display region 35A, may be displayed in the second display region 35B as one of pieces of the assistance information 44, or may be displayed in the third display region 35C.

The lesion position map 106 is a map for specifying the position of the lesion 42 in the frame 40. The lesion feature information 108 is information for specifying the kind of the lesion 42 shown in the frame 40 input to the recognition model 94 and the type of the lesion 42.

Geometric characteristics (for example, a shape and a size of an outer contour) of the lesion position map 106 correspond to geometric characteristics (for example, a shape and a size of an outer contour) of the frame 40. The lesion position map 106 is displayed on the screen 35 by the controller 82B. In the present embodiment, the lesion position map 106 is displayed in the second display region 35B by the controller 82B as one of pieces of assistance information 44. For example, the display and non-display of the lesion position map 106 are switched according to the instruction received by the reception device 64 and/or various conditions. The lesion position map 106 displayed on the screen 35 is updated according to the display frame rate applied to the first display region 35A. With such a configuration, the doctor 12 can ascertain an approximate position of the lesion 42 in the endoscopic moving image 39 displayed in the first display region 35A by referring to the lesion position map 106 displayed in the second display region 35B while observing the endoscopic moving image 39 displayed in the first display region 35A.

The controller 82B displays the lesion feature information 108 on the screen 35. For example, the lesion feature information 108 may be displayed in the second display region 35B as one of pieces of the assistance information 44, or may be displayed in the first display region 35A and/or the third display region 35C.

The recognition result 96 also includes site information 110. The site information 110 is information for specifying a site shown in the frame 40. The site information 110 may be displayed in the first display region 35A, may be displayed in the second display region 35B as one of pieces of the assistance information 44, or may be displayed in the third display region 35C.

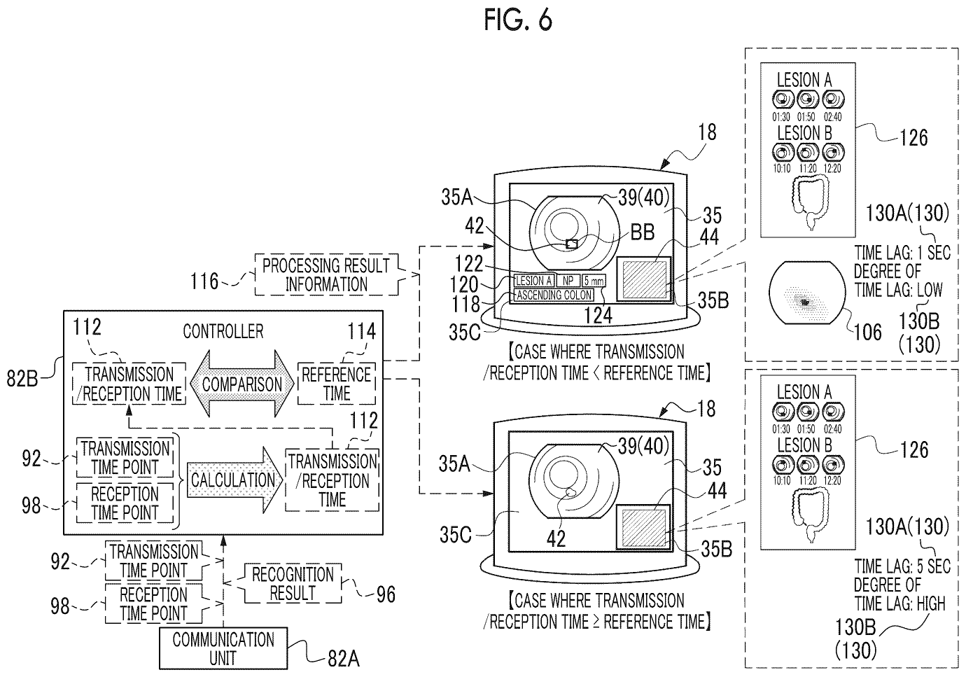

FIG. 6 is a conceptual diagram showing an example of processing contents of the communication unit 82A and the controller 82B and an example of a display content displayed on the screen 35. As shown in FIG. 6, the controller 82B calculates a transmission/reception time 112 based on the transmission time point 92 and the reception time point 98 acquired from the communication unit 82A. Here, the transmission/reception time 112 refers to a time taken from transmission of the frame 40 included in the endoscopic moving image 39 to the processing apparatus 2 by the external I/F 80 to reception of the recognition result 96 obtained by executing the recognition process 95 on the frame 40 by the external I/F 80. The transmission/reception time 112 can be said to be an indicator representing a degree of a time lag caused by transmission and reception between the medical support device 24 and the processing apparatus 2. In the present embodiment, the transmission/reception time 112 is an example of a “time” according to the present disclosure.

The controller 82B compares the transmission/reception time 112 with a reference time 114 to specify a difference between the transmission/reception time 112 and the reference time 114. The difference between the transmission/reception time 112 and the reference time 114 refers to a length of the transmission/reception time 112 with respect to the reference time 114 in other words. In the present embodiment, the reference time 114 is an example of a “first reference time”, a “second reference time”, a “third reference time”, and a “fourth reference time” according to the present disclosure.

Examples of the reference time 114 include the longest time within which a degree of affinity between the frame 40 displayed in the first display region 35A and the recognition result 96 falls within an allowable range. For example, the longest time within which the degree of affinity between the frame 40 displayed in the first display region 35A and the recognition result 96 falls within the allowable range is a time obtained in advance by a test using an actual device and/or the computer simulation or the like as the longest time recognized by the doctor 12 or the like that the content of the frame 40 displayed in the first display region 35A and the content of the recognition result 96 are consistent (that is, match).

Here, although the longest time within which the degree of affinity between the frame 40 displayed in the first display region 35A and the recognition result 96 is within the allowable range is illustrated as an example of the reference time 114, this is merely an example, and may be a statistical time (for example, a mode value, an average value, or a median value) obtained in advance by a test using an actual device and/or a computer simulation or the like as a time recognized by the doctor 12 or the like that the content of the frame 40 displayed in the first display region 35A and the content of the recognition result 96 are consistent (that is, match). In addition, the reference time 114 may be a fixed value or may be a variable value that is changed according to the instruction received by the reception device 64 or the like and/or various conditions.

The controller 82B determines whether or not to perform the display which is based on the recognition result 96, based on the transmission/reception time 112, and causes the display device 18 to perform or not to perform the display which is based on the recognition result 96, based on the determination result.

The controller 82B acquires the recognition result 96 from the processing apparatus 2, and generates and outputs processing result information 116 which is information related to the acquired recognition result 96. In the example shown in FIG. 6, outputting of the processing result information 116 by the controller 82B means that the controller 82B displays the processing result information 116 on the screen 35. The processing result information 116 includes, for example, information for specifying the lesion 42, information indicating characteristics of the lesion 42, and/or information for specifying a site where the lesion 42 is present.

The longer the transmission/reception time 112 is, the larger the degree of the time lag caused by the transmission and reception between the medical support device 24 and the processing apparatus 2 is. This means that the larger the degree of the time lag is, the higher the probability that the content of the frame 40 displayed on the screen 35 and the content of the processing result information 116 do not match, and conversely, the smaller the degree of the time lag is, the higher the probability that the content of the frame 40 displayed on the screen 35 and the content of the processing result information 116 match.

In the present embodiment, in order for the controller 82B to determine whether or not the transmission/reception time 112 which can be said to be an indicator representing the degree of the time lag is a time for which the content of the frame 40 displayed on the screen 35 and the content of the processing result information 116 match, the controller 82B compares the transmission/reception time 112 with the reference time 114 to determine whether or not the transmission/reception time 112 is equal to or longer than the reference time 114. That is, in a case where the transmission/reception time 112 is equal to or longer than the reference time 114, the controller 82B determines that the content of the frame 40 displayed on the screen 35 and the content of the processing result information 116 do not match, and in a case where the transmission/reception time 112 is shorter than the reference time 114, the controller 82B determines that the content of the frame 40 displayed on the screen 35 and the content of the processing result information 116 match. The determination result by the controller 82B is expressed by an output aspect of the processing result information 116 or is expressed by output of information related to the transmission/reception time 112.

For example, the controller 82B performs a first control of outputting the information related to the transmission/reception time 112 and a second control of changing an output aspect of the processing result information 116 according to the transmission/reception time 112.

Here, first, an example of the second control will be described. Examples of the output aspect of the processing result information 116 include a display aspect in which the controller 82B displays the processing result information 116 on the screen 35. The display aspect includes a first display aspect and a second display aspect. The first display aspect is used in a case where the transmission/reception time 112 is equal to or longer than the reference time 114 and is used to specify that the transmission/reception time 112 is equal to or longer than the reference time 114. The second display aspect is used in a case where the transmission/reception time 112 is shorter than the reference time 114 and is used to specify that the transmission/reception time 112 is shorter than the reference time 114. In the example shown in FIG. 6, in a case where the transmission/reception time 112 is equal to or longer than the reference time 114, the controller 82B displays the processing result information 116 on the screen 35 in the first display aspect, and in a case where the transmission/reception time 112 is shorter than the reference time 114, the controller 82B displays the processing result information 116 on the screen 35 in the second display aspect. The second display aspect has higher visibility than the first display aspect. For example, in the first display aspect, the processing result information 116 is not displayed, and in the second display aspect, the processing result information 116 is displayed.

In addition, the second control performed by the controller 82B includes a control of displaying the frame 40 on the screen 35 (in the example shown in FIG. 6, the first display region 35A) in a state in which the frame 40 is comparable with the processing result information 116. In the example shown in FIG. 6, in a case where the transmission/reception time 112 is shorter than the reference time 114, the processing result information 116 is displayed in the second display region 35B and the third display region 35C, and the same frame 40 as the frame 40 input to the recognition model 94 for obtaining the recognition result 96 used to generate the processing result information 116 is displayed in the first display region 35A.

Here, although a form example has been described in which the same frame 40 as the frame 40 input to the recognition model 94 for obtaining the recognition result 96 used to generate the processing result information 116 is displayed in the first display region 35A, this is merely an example. The frame 40 obtained temporally later than the frame 40 input to the recognition model 94 for obtaining the recognition result 96 used to generate the processing result information 116 (for example, the frame 40 obtained after a few frames to several tens of frames) may be displayed in the first display region 35A.

In the example shown in FIG. 6, in a case where the transmission/reception time 112 is shorter than the reference time 114, the controller 82B generates site name information 118, lesion identification information 120, discrimination information 122, and size information 124 as the processing result information 116 and displays the generated information in the third display region 35C (in the example shown in FIG. 6, a region below the first display region 35A).

The site name information 118 is information indicating a name of a site recognized by performing the recognition process 95, and is generated based on the site information 110 (see FIG. 5). Here, although the site information 110 is illustrated, information other than the site information 110 may be adopted as long as the information can specify a site where the lesion 42 is present.

The lesion identification information 120 is information for identifying the lesion 42 recognized by performing the recognition process 95. The discrimination information 122 is information indicating a result (for example, a malignancy) of discriminating the lesion 42, and is generated based on the lesion feature information 108 (see FIG. 5). The size information 124 is information indicating a size of the lesion 42 recognized by performing the recognition process 95, and is generated based on the geometric characteristic information 102 (see FIG. 5). Here, although the discrimination information 122 and the size information 124 are illustrated, information other than the discrimination information 122 and the size information 124 may be adopted as long as the information indicates the characteristics of the lesion 42.

The lesion position map 106 (see FIG. 6) includes a bounding box BB, and in a case where the transmission/reception time 112 is shorter than the reference time 114, the controller 82B displays the bounding box BB in the first display region 35A as the processing result information 116. The bounding box BB is a rectangular frame (for example, a rectangular border circumscribing an image region showing the lesion 42) for specifying a position recognized by the recognition model 94 as the position of the lesion 42 in the frame 40, which is shown in the frame 40. In the example shown in FIG. 6, the bounding box BB is displayed to be superimposed at a position where the lesion 42 in the frame 40 displayed in the first display region 35A is shown. The display of the bounding box BB is updated in synchronization with a display timing of the endoscopic moving image 39 displayed in the first display region 35A.

Here, the bounding box BB has been described, but this is merely an example. The controller 82B may display an identifier (for example, a mark defined at four corners of a rectangular frame) instead of the bounding box BB on the screen 35 (in the example shown in FIG. 6, the first display region 35A in the screen 35).

In the example shown in FIG. 6, the controller 82B generates past information 126 as one of pieces of the assistance information 44 and displays the past information 126 in the second display region 35B. The past information 126 is information generated based on the recognition result 96 obtained in the past. For example, the past information 126 includes various types of information displayed in the third display region 35C in the past, and an image, text, or the like that can specify an unobserved location or an untreated location in the current endoscopy.

Further, in a case where the transmission/reception time 112 is shorter than the reference time 114, the controller 82B displays the lesion position map 106 in the second display region 35B as one of pieces of the processing result information 116 and as one of pieces of the assistance information 44.

Here, an example of the first control will be described. The controller 82B performs a control of outputting the time information 130 related to the transmission/reception time 112 as the first control regardless of whether or not the transmission/reception time 112 is equal to or longer than the reference time 114. The time information 130 is an example of “time information” according to the present disclosure.

In the example shown in FIG. 6, an aspect in which the controller 82B displays the time information 130 in the second display region 35B is shown as an example of an aspect in which the controller 82B outputs the time information 130.

The first control performed by the controller 82B includes a control of displaying the frame 40 on the screen 35 (in the example shown in FIG. 6, the first display region 35A) in a state in which the frame 40 is comparable with the time information 130. In the example shown in FIG. 6, the time information 130 is displayed in the second display region 35B, and the same frame 40 as the frame 40 input to the recognition model 94 for obtaining the recognition result 96 received by the external I/F 80 at the reception time point 98 used to calculate the transmission/reception time 112 corresponding to the time information 130 is displayed in the first display region 35A.

Here, although a form example has been described in which the same frame 40 as the frame 40 input to the recognition model 94 for obtaining the recognition result 96 received by the external I/F 80 at the reception time point 98 used to calculate the transmission/reception time 112 corresponding to the time information 130 is displayed in the first display region 35A, this is merely an example. The frame 40 obtained temporally later than the frame 40 input to the recognition model 94 for obtaining the recognition result 96 received by the external I/F 80 at the reception time point 98 used to calculate the transmission/reception time 112 corresponding to the time information 130 (for example, the frame 40 obtained after a few frames to several tens of frames) may be displayed in the first display region 35A.

The time information 130 includes first specifying information 130A for specifying the transmission/reception time 112 itself and second specifying information 130B for specifying a difference between the transmission/reception time 112 and the reference time 114. The first specifying information 130A and the second specifying information 130B are information visualized by a text. The first specifying information 130A is an example of “first specifying information” according to the present disclosure, and the second specifying information 130B is an example of “second specifying information” according to the present disclosure.

In the example shown in FIG. 6, the transmission/reception time 112 itself is displayed in the second display region 35B in a text format as the first specifying information 130A. In addition, the first specifying information 130A is displayed in the second display region 35B in a content that the first specifying information 130A can be specified as information indicating a time lag (that is, a communication delay time).

In the example shown in FIG. 6, length information for specifying a length of the transmission/reception time 112 with respect to the reference time 114 is displayed in the second display region 35B in a text format as the second specifying information 130B. Examples of the length information include information for specifying whether or not the transmission/reception time 112 is equal to or longer than the reference time 114. In the example shown in FIG. 6, in a case where the transmission/reception time 112 is equal to or longer than the reference time 114, the text “high” is displayed as the length information, and in a case where the transmission/reception time 112 is shorter than the reference time 114, the text “low” is displayed as the length information. In addition, the second specifying information 130B is displayed in the second display region 35B in a content that the second specifying information 130B can be specified as information indicating the degree of the time lag. Here, the text “high” and the text “low” are examples of “length information” according to the present disclosure.

In addition, here, although the text “high” and the text “low” are illustrated as the length information, this is merely an example. In a case where the transmission/reception time 112 is equal to or longer than the reference time 114, any information for specifying that the transmission/reception time 112 is equal to or longer than the reference time 114 may be output (for example, an indicator for specifying that the transmission/reception time 112 is equal to or longer than the reference time 114) may be output, and in a case where the transmission/reception time 112 is shorter than the reference time 114, any information for specifying that the transmission/reception time 112 is shorter than the reference time 114 (for example, an indicator for specifying that the transmission/reception time 112 is shorter than the reference time 114) may be output.

In a case where the transmission/reception time 112 is shorter than the reference time 114, any information for specifying that the transmission/reception time 112 is shorter than the reference time 114 may not be output. The reason for this is that, in a case where the transmission/reception time 112 is shorter than the reference time 114, the content of the frame 40 displayed in the first display region 35A matches the processing result information 116, and there is no particular problem for the doctor 12 to perform the endoscopy even though the time information 130 is not output.

The controller 82B changes a visualization format, which is a format for visualizing the time information 130, according to the transmission/reception time 112. For example, the controller 82B makes the visibility of the time information 130 in a case where the transmission/reception time 112 is equal to or longer than the reference time 114 higher than the visibility of the time information 130 in a case where the transmission/reception time 112 is shorter than the reference time 114. In the example shown in FIG. 6, the controller 82B displays the time information 130 at different visibility levels between a case where the transmission/reception time 112 is shorter than the reference time 114 and a case where the transmission/reception time 112 is equal to or longer than the reference time 114. For example, the controller 82B displays the time information 130 in a case where the transmission/reception time 112 is shorter than the reference time 114 in a first visibility level, and displays the time information 130 in a case where the transmission/reception time 112 is equal to or longer than the reference time 114 in a second visibility level.

The second visibility level is a visibility level higher than the first visibility level. For example, a height of the visibility level is determined by a size of the text of the time information 130. In the example shown in FIG. 6, the size of the text of the time information 130 at the second visibility level is larger than the size of the text of the time information 130 at the first visibility level. That is, the controller 82B increases the size of the text of the time information 130 in a case where the transmission/reception time 112 is equal to or longer than the reference time 114 as compared with the size of the text of the time information 130 in a case where the transmission/reception time 112 is shorter than the reference time 114. As a result, the visibility of the time information 130 in a case where the transmission/reception time 112 is equal to or longer than the reference time 114 is higher than the visibility of the time information 130 in a case where the transmission/reception time 112 is shorter than the reference time 114.

Here, although a form example in which the visibility level is made different by changing the size of the text of the time information 130 is illustrated, this is merely an example. For example, the visibility level may be made different by changing a transparency of the text of the time information 130, a color density of the text of the time information 130, and/or a brightness of the text of the time information 130. In addition, in a case where the transmission/reception time 112 is equal to or longer than the reference time 114, the time information 130 may be displayed, and in a case where the transmission/reception time 112 is shorter than the reference time 114, the time information 130 may not be displayed, so that the visibility of the time information 130 may be made different.

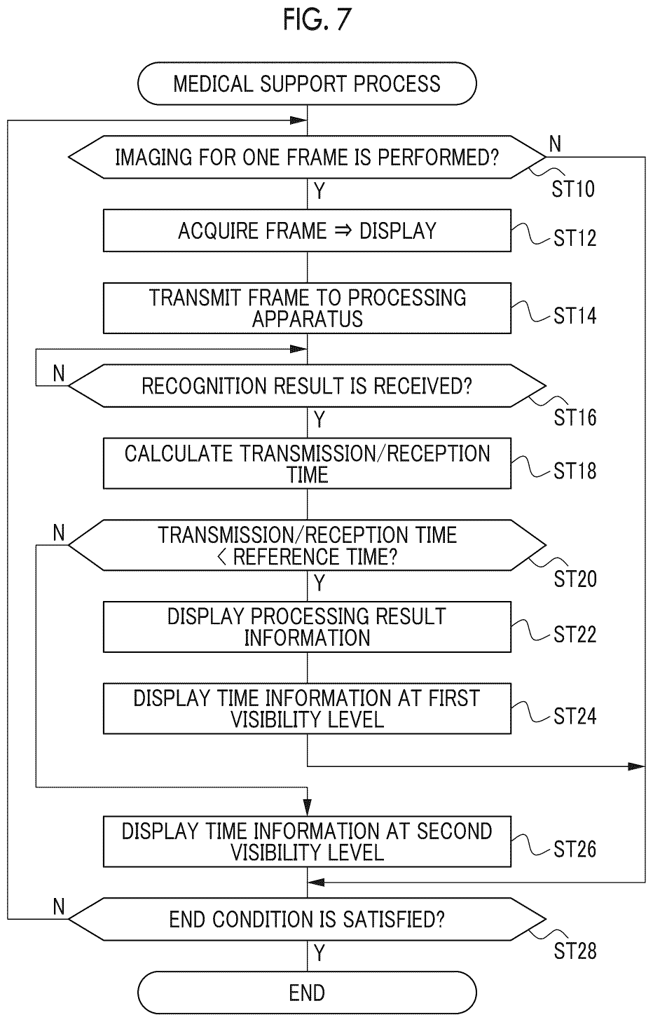

Next, an operation of a part of the medical support system 1 according to the embodiment of the present disclosure will be described with reference to FIG. 7. A flow of the medical support process shown in FIG. 7 is an example of a “medical support method” according to the present disclosure.

In the medical support process shown in FIG. 7, first, in step ST10, the controller 82B determines whether or not imaging for one frame is performed by the camera 52 in the large intestine 28. In step ST10, in a case where the imaging for one frame is not performed by the camera 52 in the large intestine 28, a negative determination is made, and the medical support process proceeds to step ST28. In step ST10, in a case where the imaging for one frame is performed by the camera 52 in the large intestine 28, a positive determination is made, and the medical support process proceeds to step ST12.

In step ST12, the communication unit 82A and the controller 82B acquire the frame 40 obtained by imaging the inside of the large intestine 28 (for example, the intestinal wall 32) by the camera 52. The controller 82B displays the frame 40 in the first display region 35A. In a case where the frame 40 is already displayed in the first display region 35A, the controller 82B updates the frame 40 displayed in the first display region 35A. That is, by repeatedly executing the process in step ST12, the frame 40 is displayed in the first display region 35A by the live view method. After the process in step ST12 is executed, the medical support process proceeds to step ST14.

In step ST14, the communication unit 82A causes the external I/F 80 to transmit the frame 40 acquired in step ST12 to the processing apparatus 2. Moreover, the communication unit 82A acquires the transmission time point 92. After the process in step ST14 is executed, the medical support process proceeds to step ST16.

In a case where the process of step ST14 is executed to transmit the frame 40 to the processing apparatus 2, the processing apparatus 2 executes the recognition process 95 on the frame 40 and transmits the recognition result 96 of the recognition process 95 to the medical support device 24.

In step ST16, the communication unit 82A determines whether or not the recognition result 96 is received by the external I/F 80. In step ST16, in a case where the recognition result 96 is not received by the external I/F 80, a negative determination is made, and the determination in step ST16 is made again. In step ST16, in a case where the recognition result 96 is received by the external I/F 80, a positive determination is made, and the medical support process proceeds to step ST18. In addition, in step ST16, in a case where the recognition result 96 is received by the external I/F 80, the communication unit 82A acquires the reception time point 98.

In step ST18, the controller 82B acquires the transmission time point 92 and the reception time point 98 from the communication unit 82A and calculates the transmission/reception time 112 based on the transmission time point 92 and the reception time point 98. After the process in step ST18 is executed, the medical support process proceeds to step ST20.

In step ST20, the controller 82B determines whether or not the transmission/reception time 112 calculated in step ST18 is shorter than the reference time 114. In step ST20, in a case where the transmission/reception time 112 calculated in step ST18 is not shorter than the reference time 114, a negative determination is made, and the medical support process proceeds to step ST26. In step ST20, in a case where the transmission/reception time 112 calculated in step ST18 is shorter than the reference time 114, a positive determination is made, and the medical support process proceeds to step ST22.

In step ST22, the controller 82B displays the processing result information 116 on the screen 35. After the process in step ST22 is executed, the medical support process proceeds to step ST24.

In step ST24, the controller 82B displays the time information 130 in the second display region 35B at the first visibility level. After the process in step ST24 is executed, the medical support process proceeds to step ST28.

In step ST26, the controller 82B displays the time information 130 in the second display region 35B at the second visibility level. After the process in step ST26 is executed, the medical support process proceeds to step ST28.

In step ST28, the controller 82B determines whether or not a medical support process end condition is satisfied. An example of the medical support process end condition is a condition that an instruction for the medical support system 1 to end the medical support process is given (for example, a condition that the reception device 64 receives an instruction to end the medical support process).

In a case where the medical support process end condition is not satisfied in step ST28, a negative determination is made, and the medical support process proceeds to step ST10. In a case where the medical support process end condition is satisfied in step ST28, a positive determination is made, and the medical support process ends.

As described above, in the medical support system 1, the time information 130 is displayed on the screen 35. The time information 130 is information related to the transmission/reception time 112 required from when the frame 40 is transmitted from the external I/F 80 of the medical support device 24 to the processing apparatus 2 to when the recognition result 96 obtained by executing the recognition process 95 on the frame 40 by the processing apparatus 2 is received by the external I/F 80 of the medical support device 24. The transmission/reception time 112 can be said to be an indicator representing a degree of a time lag caused by transmission and reception between the medical support device 24 and the processing apparatus 2. Therefore, in the medical support system 1, the display aspect of the processing result information 116 is made different depending on the transmission/reception time 112. In this way, the doctor 12 can visually recognize the degree of the time lag caused by transmission and reception between the medical support device 24 and the processing apparatus 2 from the display aspect of the time information 130 and/or the processing result information 116 displayed on the screen 35.

In addition, in the medical support system 1, the time information 130 and the frame 40 are displayed on the screen 35 in a state in which the time information 130 and the frame 40 are comparable. Therefore, the doctor 12 can determine whether or not the content of the frame 40 and the processing result information 116 match each other while referring to the time information 130.

In addition, in the medical support system 1, the first specifying information 130A is displayed in the second display region 35B. The first specifying information 130A is information for specifying the transmission/reception time 112 itself. Therefore, the doctor 12 can visually recognize the transmission/reception time 112 itself which indicates the degree of the time lag caused by the transmission and reception between the medical support device 24 and the processing apparatus 2.

In addition, in the medical support system 1, the second specifying information 130B is displayed in the second display region 35B. The second specifying information 130B is information for specifying a difference between the transmission/reception time 112 and the reference time 114 (that is, a length of the transmission/reception time 112 with respect to the reference time 114). Therefore, the doctor 12 can visually recognize the difference between the transmission/reception time 112 and the reference time 114 (that is, the length of the transmission/reception time 112 with respect to the reference time 114) indicating the degree of the time lag caused by the transmission and reception between the medical support device 24 and the processing apparatus 2.

In addition, in the medical support system 1, the time information 130 is displayed in the second display region 35B as visualized information. For example, the time information 130 is visualized by a text. Therefore, the degree of the time lag caused by the transmission and reception between the medical support device 24 and the processing apparatus 2 is visualized to be perceived by the doctor 12.

In addition, in the medical support system 1, a visualization format is changed according to the transmission/reception time 112. For example, the size of the text of the time information 130 is changed according to the transmission/reception time 112. In this case, for example, the controller 82B increases the size of the text of the time information 130 displayed in the second display region 35B in a case where the transmission/reception time 112 is equal to or longer than the reference time 114 as compared with the size of the text of the time information 130 in a case where the transmission/reception time 112 is shorter than the reference time 114. Accordingly, the doctor 12 can visually recognize whether or not the transmission/reception time 112 is equal to or longer than the reference time 114.

In addition, in the medical support system 1, the display aspect of the processing result information 116 is changed according to whether or not the transmission/reception time 112 is equal to or longer than the reference time 114. Therefore, the doctor 12 can determine whether or not the transmission/reception time 112 is equal to or longer than the reference time 114 from the display aspect of the processing result information 116.

In addition, in the medical support system 1, the processing result information 116 is displayed on the screen 35 in a case where the transmission/reception time 112 is shorter than the reference time 114, and the processing result information 116 is not displayed on the screen 35 in a case where the transmission/reception time 112 is equal to or longer than the reference time 114. As a result, the doctor 12 can visually distinguish between a case where the transmission/reception time 112 is shorter than the reference time 114 and a case where the transmission/reception time 112 is equal to or longer than the reference time 114. In addition, since the processing result information 116 is not displayed on the screen 35, the doctor 12 can recognize that the content of the frame 40 and the processing result information 116 do not match.

In addition, in the medical support system 1, the frame 40 and the processing result information 116 are displayed on the screen 35 in a state in which the frame 40 and the processing result information 116 are comparable. Therefore, the doctor 12 can determine whether or not the frame 40 and the processing result information 116 match each other.

In addition, in the medical support system 1, in a case where the transmission/reception time 112 is shorter than the reference time 114, the frame 40 and the processing result information 116 are displayed on the screen 35 in a state in which the frame 40 and the processing result information 116 are comparable. Therefore, the doctor 12 can visually ascertain that the transmission/reception time 112 is shorter than the reference time 114 and can determine whether or not the frame 40 and the processing result information 116 match each other.

In addition, in the medical support system 1, as the reference time 114 to be compared with the transmission/reception time 112 by the controller 82B, the longest time within which the degree of affinity between the frame 40 displayed in the first display region 35A and the recognition result 96 falls within the allowable range is used. Therefore, as compared with a case where a time within which the degree of affinity between the frame 40 displayed in the first display region 35A and the recognition result 96 does not fall within the allowable range is used as the reference time 114, it is possible to prevent the doctor 12 from erroneously recognizing that the frame 40 currently displayed in the first display region 35A and the processing result information 116 displayed on the screen 35 match each other even though the recognition result 96 that does not match the frame 40 currently displayed in the first display region 35A is obtained due to the excessively long time lag.

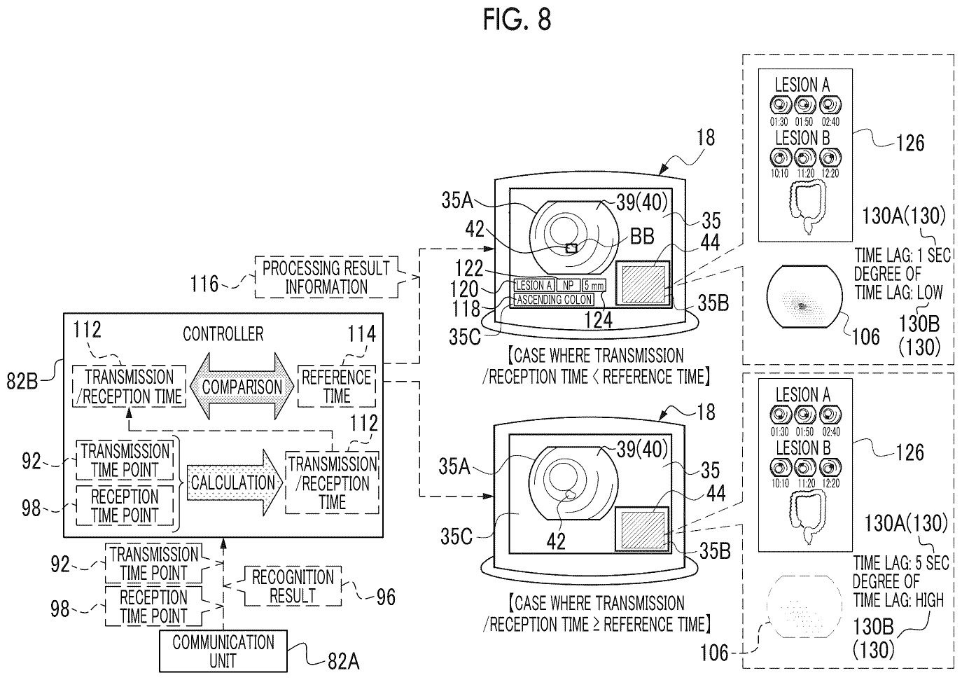

In the embodiment described above, although a form example has been described in which the lesion position map 106 is displayed in the second display region 35B as one of pieces of the processing result information 116 and as one of pieces of the assistance information 44 in a case where the transmission/reception time 112 is shorter than the reference time 114, the present disclosure is not limited to this. For example, as shown in FIG. 8, the lesion position map 106 may be displayed in the second display region 35B regardless of whether or not the transmission/reception time 112 is shorter than the reference time 114.

The consistency between the recognition result 96 and the frame 40 displayed in the first display region 35A in a case where the transmission/reception time 112 is equal to or longer than the reference time 114 is lower than the consistency between the recognition result 96 and the frame 40 displayed in the first display region 35A in a case where the transmission/reception time 112 is shorter than the reference time 114. Therefore, in order for the doctor 12 to visually recognize this fact, the controller 82B makes the visibility of the lesion position map 106 displayed in the second display region 35B in a case where the transmission/reception time 112 is equal to or longer than the reference time 114 lower than the visibility of the lesion position map 106 displayed in the second display region 35B in a case where the transmission/reception time 112 is shorter than the reference time 114. As a result, the doctor 12 can visually recognize whether or not the degree of the time lag is equal to or greater than the reference time 114 from the visibility of the lesion position map 106. In addition, the doctor 12 can visually ascertain a degree of consistency between the recognition result 96 and the frame 40 displayed in the first display region 35A from the visibility of the lesion position map 106.

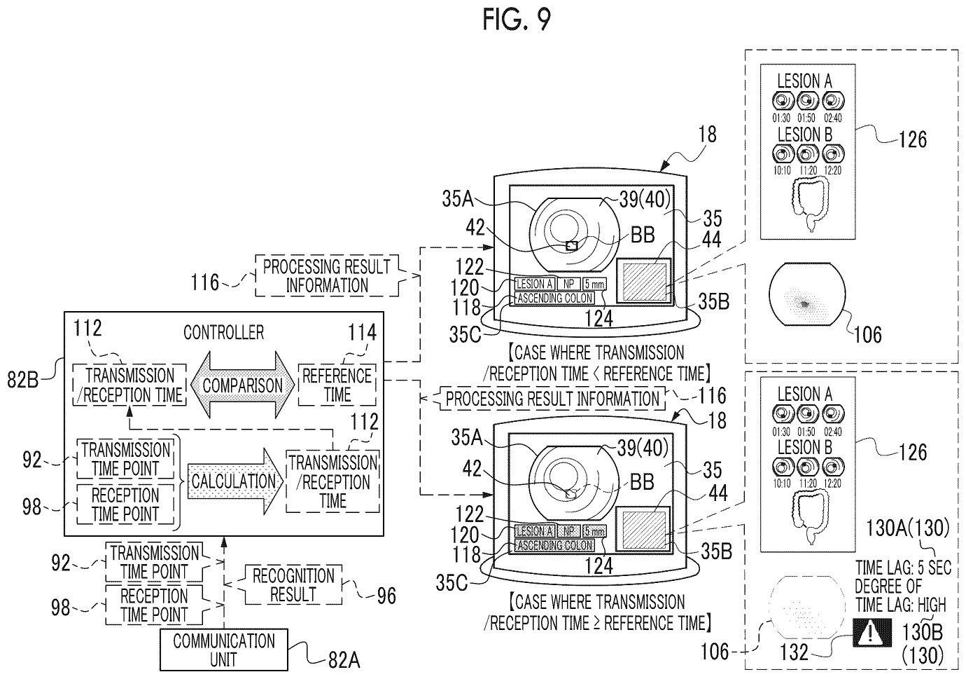

In the embodiment described above, although a form example has been illustrated in which the processing result information 116 is not displayed on the screen 35 in a case where the transmission/reception time 112 is equal to or longer than the reference time 114, this is merely an example. For example, as shown in FIG. 9, even in a case where the transmission/reception time 112 is equal to or longer than the reference time 114, the processing result information 116 may be displayed on the screen 35. In this case, the visibility of the processing result information 116 is reduced. That is, in a case where the transmission/reception time 112 is equal to or longer than the reference time 114, the visibility of the processing result information 116 displayed on the screen 35 is lower than that in a case where the transmission/reception time 112 is shorter than the reference time 114. In the example shown in FIG. 9, in a case where the transmission/reception time 112 is equal to or longer than the reference time 114, the site name information 118, the lesion identification information 120, the discrimination information 122, and the size information 124 are displayed in a grayed-out state in the third display region 35C, and a line of the bounding box BB is displayed to be thinner than a line of the bounding box BB displayed in a case where the transmission/reception time 112 is shorter than the reference time 114. There are various methods for reducing the visibility. For example, there are a method of reducing visibility by increasing a transparency of the display, a method of reducing visibility by covering a display location with some kind of mask, and a method of reducing visibility by blinking.

In addition, as shown in FIG. 9 as an example, in a case where the transmission/reception time 112 is shorter than the reference time 114, the time information 130 may not be displayed on the screen 35. By not displaying the time information 130 on the screen 35, the doctor 12 can proceed with a medical process without paying attention to the time information 130.

Further, as shown in FIG. 9 as an example, in a case where the transmission/reception time 112 is equal to or longer than the reference time 114, a notification mark 132 may be displayed on the screen 35 (in the example shown in FIG. 9, the second display region 35B) as one of pieces of information in which the time information 130 is visualized. The notification mark 132 is a mark for notifying that the transmission/reception time 112 is equal to or longer than the reference time 114 (in other words, the processing result information 116 displayed on the screen 35 and the content of the frame 40 displayed in the first display region 35A do not match). In this way, by displaying the notification mark 132 on the screen 35, the doctor 12 can visually ascertain, from the notification mark 132, that the transmission/reception time 112 is equal to or longer than the reference time 114 and that the processing result information 116 displayed on the screen 35 and the content of the frame 40 displayed in the first display region 35A do not match. The notification mark 132 is an example of “time information” and a “mark” according to the present disclosure.

In the example shown in FIG. 9, in a case where the transmission/reception time 112 is equal to or longer than the reference time 114, the visibility of the bounding box BB displayed on the screen 35 is lower than that in a case where the transmission/reception time 112 is shorter than the reference time 114. However, this is merely an example, and the display aspect (for example, a color, a line type, or a shape) of the bounding box BB may be made different between a case where the transmission/reception time 112 is equal to or longer than the reference time 114 and a case where the transmission/reception time 112 is shorter than the reference time 114. In addition, the display aspect (for example, a color, a line type, or a shape) of the bounding box BB may be made different depending on the kind and/or the type of the lesion 42.

In the embodiment described above, although a form example has been described in which the time information 130 and the processing result information 116 are displayed on the screen 35, this is merely an example, and the time information 130 and/or the processing result information 116 may be output as a voice from a speaker (not shown). In this case, for example, the controller 82B may adjust a sound quality and/or a volume or like of the voice output from the speaker to make the voice in a case where the transmission/reception time 112 is equal to or longer than the reference time 114 easier to hear than the voice in a case where the transmission/reception time 112 is shorter than the reference time 114.

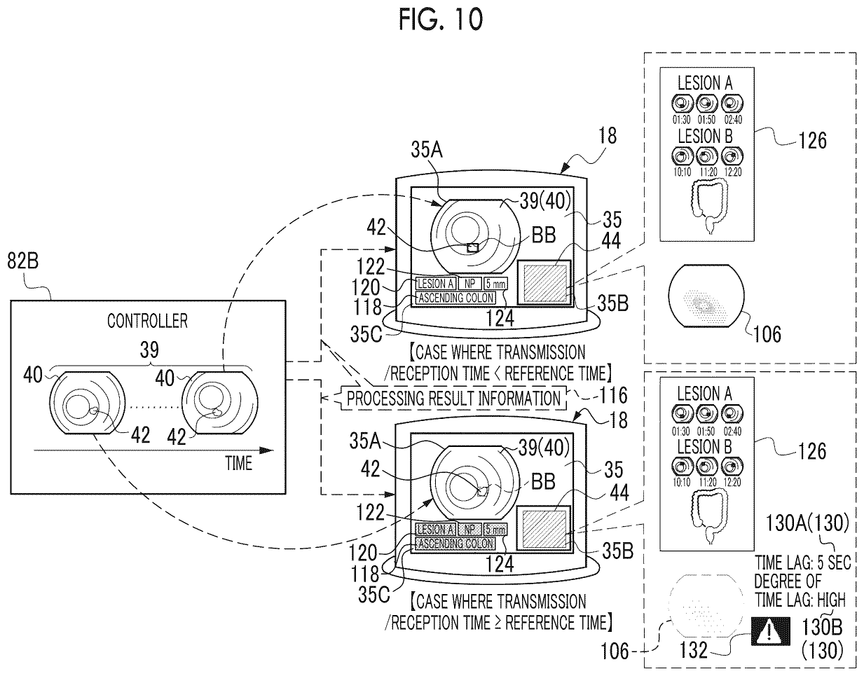

In the embodiment described above, a form example has been described in which it is premised that the frame 40 displayed in the first display region 35A in a case where the transmission/reception time 112 is shorter than the reference time 114 and the frame 40 displayed in the first display region 35A in a case where the transmission/reception time 112 is equal to or longer than the reference time 114 are the same, but the present disclosure is not limited to this. For example, as shown in FIG. 10, the controller 82B may use, as the frame 40 displayed in the first display region 35A in a case where the transmission/reception time 112 is equal to or longer than the reference time 114, the frame 40 obtained temporally later than the frame 40 displayed in the first display region 35A in a case where the transmission/reception time 112 is shorter than the reference time 114. Here, the frame 40 displayed in the first display region 35A in a case where the transmission/reception time 112 is equal to or longer than the reference time 114 is an example of a “second medical image” and a “third medical image” according to the present disclosure.

In this way, by using the frame 40 obtained temporally later than the frame 40 displayed in the first display region 35A in a case where the transmission/reception time 112 is shorter than the reference time 114 as the frame 40 displayed in the first display region 35A in a case where the transmission/reception time 112 is equal to or longer than the reference time 114, it is possible to make the content of the frame 40 displayed in the first display region 35A and the processing result information 116 displayed on the screen 35 match each other even in a case where the transmission/reception time 112 is equal to or longer than the reference time 114.

In addition, in a case where the transmission/reception time 112 is equal to or longer than the reference time 114, guide information recommending that the camera 52 inserted into the large intestine 28 be made stationary or an insertion or removal speed of the camera 52 be slowed may be displayed on the screen 35. In this way, the content of the frame 40 displayed in the first display region 35A and the processing result information 116 displayed on the screen 35 can be matched with each other.

In the embodiment described above, although a form example has been described in which the time information 130 is displayed in the second display region 35B, this is merely an example, and the time information 130 may be displayed in a region other than the second display region 35B. The same applies to the processing result information 116.

In addition, the processing result information 116 and the time information 130 do not need to be displayed only on the screen 35 of the display device 18. For example, the processing result information 116 may be displayed in a distributed manner on a plurality of display devices, and the time information 130 may also be displayed in a distributed manner on a plurality of display devices.

In the embodiment described above, the lesion 42 has been illustrated as an example of a “feature region” according to the present disclosure, but the present disclosure is not limited to this. Even in a case where a resection region, a bleeding region, a marking region, an organ, a treatment tool (for example, a hemostatic clip placed in the body), or the like is applied instead of the lesion 42, the technology of the present disclosure is established.

In the embodiment described above, although the camera 52 mounted on the endoscope 16 is illustrated, the present disclosure is not limited to this. For example, the technology of the present disclosure can be applied to a fundus camera, a camera mounted on a medical microscope, or the like.

In the embodiment described above, the frame 40 obtained by imaging the inside of the large intestine 28 by the camera 52 mounted on the endoscope 16 has been illustrated. However, this is merely an example, and the frame 40 may be a medical image obtained by various modalities such as a radiography apparatus, an MRI, a CT, or an ultrasound probe.

In the embodiment described above, the recognition model 94 has been illustrated, but a so-called generation AI may be used as the recognition model 94. For example, instruction data (so-called prompt) including the frame 40 may be input to the generation AI, and the recognition result 96 or the processing result information 116 may be output from the generation AI. In addition, the generation AI may output the assistance information 44 according to the input prompt in addition to the recognition result 96 or the processing result information 116. Examples of the generation AI include ChatGPT using GPT-4 (Internet search <https://openai.com/gpt-4>).

Instruction data including at least a part of the processing result information 116 may be used as the input information for the generation AI. Examples of the information output from the generation AI include information related to an operation content of the camera 52, information indicating a content of a medical treatment that is recommended to be performed during the endoscopy, and/or information indicating a content of a medical treatment that is recommended to be performed after the endoscopy. The information output from the generation AI may be stored in various storage regions (for example, the storage 76 and/or 86), displayed on the display device 18 as the assistance information 44, printed on a medium by the printer, or output from a speaker as a voice.

In the embodiment described above, the recognition process 95 using AI in a bounding box method has been illustrated, but this is merely an example. For example, a recognition process using AI in a segmentation method may be performed instead of the recognition process 95 using AI in a bounding box method. In addition, a recognition process in a non-AI method (for example, a template matching method) may be performed instead of the recognition process in an AI method, or a recognition process in which the non-AI method and the AI method are combined may be performed.

In the embodiment described above, a form example is described in which the medical support process is performed by the computer 78, but the present disclosure is not limited to this. At least some of processing included in the medical support process may be performed by a device provided outside the computer 78.

In the embodiment described above, although a form example has been described in which the processing apparatus 2 is realized by cloud computing, this is merely an example. The processing apparatus 2 may be realized by network computing such as fog computing, edge computing, or grid computing.

In the embodiment described above, a form example is described in which the medical support program 90 is stored in the storage 86, but the present disclosure is not limited to this. For example, the medical support program 90 may be stored in a portable computer-readable non-transitory storage medium, such as an SSD or a USB memory. The medical support program 90 stored in the non-transitory storage medium is installed in the computer 78 of the endoscope apparatus 10. The processor 82 executes the medical support process according to the medical support program 90.

In addition, the medical support program 90 may be stored in a storage device of another computer, server, or the like connected to the endoscope apparatus 10 via a network, and the medical support program 90 may be downloaded and installed in the computer 78 in response to a request from the endoscope apparatus 10.

It is not necessary to store all the medical support programs 90 in a storage device of another computer, server device, or the like connected to the endoscope apparatus 10 or to store all the medical support programs 90 in the storage 86, and a part of the medical support programs 90 may be stored.

The following various processors can be used as hardware resources for executing the medical support process. Examples of the processor include a CPU which is a general-purpose processor that executes software, that is, a program, to function as the hardware resource executing the medical support process. In addition, examples of the processor include a dedicated electric circuit which is a processor having a circuit configuration specially designed for executing specific processing, such as an FPGA, a PLD, or an ASIC. A memory is incorporated in or connected to any processor, and any processor executes the medical support process using the memory.

The hardware resource for executing the medical support process may be configured by one of the various processors or by a combination of two or more processors of the same type or different types (for example, a combination of a plurality of FPGAs or a combination of a CPU and an FPGA). Further, the hardware resource for executing the medical support process may be one processor.

As an example of the configuration using one processor, first, there is a form in which one processor is configured by a combination of one or more CPUs and software, and the processor functions as the hardware resource for executing the medical support process. Second, as typified by a SoC or the like, there is a form in which a processor that realizes all functions of a system including a plurality of hardware resources executing the medical support process with one IC chip is used. As described above, the medical support process is realized using one or more of the various processors as the hardware resource.

Further, as a hardware structure of these various processors, more specifically, an electrical circuit in which circuit elements such as semiconductor elements are combined can be used. Further, the above-described medical support process is only an example. Therefore, it is needless to say that unnecessary steps may be deleted, new steps may be added, or a processing order may be changed without departing from the gist of the present disclosure.

The above-described contents and illustrated contents are detailed descriptions of parts related to the present disclosure, and are merely examples of the present disclosure. For example, the above descriptions related to configurations, functions, operations, and advantageous effects are descriptions related to examples of configurations, functions, operations, and advantageous effects of the parts related to the present disclosure. Therefore, it is needless to say that unnecessary parts may be deleted, or new elements may be added or replaced with respect to the above-described contents and illustrated contents without departing from the gist of the present disclosure. In order to avoid complications and easily understand the parts according to the present disclosure, in the above-described contents and illustrated contents, common technical knowledge and the like that do not need to be described to implement the present disclosure are not described.

All documents, patent applications, and technical standards described in the present specification are incorporated in the present specification by reference to the same extent as in a case where each document, patent application, and technical standard are specifically and individually noted to be incorporated by reference.

In regard to the embodiment described above, the following appendices will be further disclosed.

### APPENDIX 1

A medical support device comprising: a communication device; and a processor, in which the communication device transmits a medical image to a processing apparatus, and receives a processing result obtained by executing processing on the medical image by the processing apparatus, and the processor performs a first control of outputting time information related to a time taken from transmission of the medical image to the processing apparatus by the communication device to reception of the processing result, and/or a second control of making an output aspect of the processing result different depending on the time.

### APPENDIX 2

The medical support device according to Appendix 1, in which the outputting of the time information by the processor includes displaying the time information on a first screen by the processor.

### APPENDIX 3

The medical support device according to Appendix 2, in which the first control includes a control of displaying the medical image on a screen in a state in which the medical image is comparable with the time information in a case where the time is shorter than a reference time.

As a result, a user (for example, a doctor) can easily ascertain a relationship between the medical image processed by the processing apparatus and a time lag generated by transmission and reception.

### APPENDIX 4

The medical support device according to Appendix 2, in which the first control includes a control of displaying the medical image on a second screen in a state in which the medical image is comparable with the time information in a case where the time is equal to or longer than a reference time.

As a result, a user (for example, a doctor) can easily ascertain a relationship between the medical image processed by the processing apparatus and a time lag generated by transmission and reception.

What is claimed is:

1. A medical support device comprising:
a communication device; and
a processor,
wherein the communication device is configured to transmit a first medical image to a processing apparatus, and receive a processing result obtained by executing processing on the first medical image by the processing apparatus, and
the processor is configured to perform a first control of outputting time information related to a time taken from transmission of the first medical image to the processing apparatus by the communication device to reception of the processing result, and/or a second control of making an output aspect of the processing result different depending on the time.

2. The medical support device according to claim 1,
wherein the outputting of the time information by the processor includes displaying the time information on a first screen by the processor.

3. The medical support device according to claim 2,
wherein the first control includes a control of displaying the first medical image and/or a second medical image obtained temporally later than the first medical image on a second screen in a state in which the first medical image and/or the second medical image is comparable with the time information.

4. The medical support device according to claim 1,
wherein the time information includes first specifying information for specifying the time itself.

5. The medical support device according to claim 1,
wherein the time information includes second specifying information for specifying a difference between the time and a first reference time.

6. The medical support device according to claim 5,
wherein the second specifying information includes length information for specifying a length of the time with respect to the first reference time.

7. The medical support device according to claim 1,
wherein the time information is visualized information.

8. The medical support device according to claim 7,
wherein the time information is information visualized by a text and/or a mark.

9. The medical support device according to claim 7,
wherein the processor changes a visualization format, which is a format in which the time information is visualized, according to the time.

10. The medical support device according to claim 9,
wherein the processor is configured to make visibility of the time information in a case where the time is equal to or longer than a second reference time higher than visibility of the time information in a case where the time is shorter than the second reference time.

11. The medical support device according to claim 1,
wherein the output aspect is a display aspect in which the processor displays the processing result on a third screen.

12. The medical support device according to claim 11,
wherein the display aspect includes a first display aspect and a second display aspect,
the first display aspect is used in a case where the time is equal to or longer than a third reference time and is used to specify that the time is equal to or longer than the third reference time, and
the second display aspect is used in a case where the time is shorter than the third reference time and is used to specify that the time is shorter than the third reference time.

13. The medical support device according to claim 12,
wherein the second display aspect has higher visibility than the first display aspect.

14. The medical support device according to claim 13,
wherein the processing result is not displayed in the first display aspect, and
the processing result is displayed in the second display aspect.

15. The medical support device according to claim 11,
wherein the second control includes a control of displaying the first medical image and/or a third medical image obtained temporally later than the first medical image on a fourth screen in a state in which the first medical image and/or the third medical image is comparable with the processing result.

16. The medical support device according to claim 11,
wherein the second control includes a control of displaying the first medical image on a fourth screen in a state in which the first medical image is comparable with the processing result in a case where the time is shorter than a fourth reference time.

17. The medical support device according to claim 16,
wherein the fourth reference time is a longest time within which a degree of affinity between the first medical image and the processing result falls within an allowable range.

18. The medical support device according to claim 1,
wherein the processing is an AI process for the first medical image.

19. The medical support device according to claim 1,
wherein the processing includes a recognition process of recognizing a feature region shown in the first medical image.

20. The medical support device according to claim 19,
wherein the feature region is a lesion.

21. The medical support device according to claim 1,
wherein the first medical image is an endoscopic image generated by imaging an inside of a body by a camera mounted on an endoscope.

22. An endoscope apparatus comprising:
the medical support device according to claim 1; and
a camera that is inserted into a body and generates the first medical image by imaging an inside of the body.

23. A medical support system comprising:
a medical support device; and
a processing apparatus,
wherein the medical support device is configured to transmit a first medical image to the processing apparatus,
the processing apparatus is configured to execute processing on the first medical image and transmit a processing result obtained by executing the processing to the medical support device, and
the medical support device is configured to receive the processing result transmitted from the processing apparatus, and perform a first control of outputting time information related to a time taken from transmission of the first medical image to the processing apparatus to reception of the processing result by the medical support device, and/or a second control of making an output aspect of the processing result different depending on the time.

24. A medical support method comprising:
transmitting a first medical image to a processing apparatus;
receiving a processing result obtained by executing processing on the first medical image by the processing apparatus; and
performing a first control of outputting time information related to a time taken from transmission of the first medical image to the processing apparatus to reception of the processing result, and/or a second control of making an output aspect of the processing result different depending on the time.

25. A non-transitory computer-readable storage medium storing a program executable by a computer to execute a medical support process comprising:
transmitting a first medical image to a processing apparatus;
receiving a processing result obtained by executing processing on the first medical image by the processing apparatus; and
performing a first control of outputting time information related to a time taken from transmission of the first medical image to the processing apparatus to reception of the processing result, and/or a second control of making an output aspect of the processing result different depending on the time.
